(19) United States
(12) Patent Application Publication
(10) Pub. No.: US 2007/0092532 A1
(43) Pub. Date: Apr. 26, 2007

(54) Arthritis vaccines and methods

(75) Inventor: Robert S. Root-Bernstein, East Lansing, MI (US)

Correspondence Address: HARNESS, DICKEY & PIERCE, P.L.C., P.O. BOX 828, BLOOMFIELD HILLS, MI, 48303, US

(73) Assignee: Board of Trustees Operating Michigan State University, East Lansing, MI (US)

(21) Appl. No.: 11/586,346

(22) Filed: Oct. 25, 2006

Related U.S. Application Data
(60) Provisional application No. 60/730,090, filed on Oct. 25, 2005.

(51) Int. Cl. A61K 48/00; A61K 39/00

(52) U.S. Cl. 424/185.1; 514/44

(57) ABSTRACT

Vaccine compositions for preventing the onset or induction of arthritis in a subject susceptible to developing arthritis, comprising an arthritic protective peptide comprising the amino acid sequence of SEQ ID NO: 1. In various embodiments, the anti-arthritic peptide is admixed with an adjuvant in a pharmaceutically acceptable carrier. Methods include preventing arthritis in a human or other mammal subject comprising administering the arthritic protective peptide to the subject.

### CROSS REFERENCE TO RELATED APPLICATIONS

This application claims the benefit of U.S. Provisional Application No. 60/730,090, filed Oct. 25, 2005, the entire contents of which are hereby incorporated by reference into the present application.

### BACKGROUND

The present invention relates to vaccine compositions to prevent or delay the onset of arthritis in subjects and methods of use thereof. More particularly, the present invention relates to arthritis vaccines comprising anti-arthritic peptides, methods of administering such vaccines and kits.

Inflammation occurs when tissues are injured by viruses, bacteria, trauma, chemicals, heat, cold or any other harmful stimulus. Chemicals including bradykinin, histamine, serotonin and others are released, attracting tissue macrophages and white blood cells to localize in an area to engulf and destroy foreign substances. During this process, chemical mediators such as TNFα are released, giving rise to inflammation. Inflammatory disorders are those in which the inflammation is sustained or chronic. One group of inflammatory disorders are immunoinflammatory disorders such as rheumatoid arthritis (RA), osteoarthritis, psoriasis, ulcerative colitis, Crohn's disease, ankylosing spondylitis, fibromyalgia, gout, systemic lupus erythematosus, scleroderma, and Sjögren's syndrome, which are characterized by dysregulation of the immune system and inappropriate mobilization of body's defenses against its own healthy tissue.

One percent of people in the United States are afflicted with RA, a relentless, progressive disease causing severe swelling, pain, and eventual deformity and destruction of joints. Rheumatic diseases are the leading cause of disability among adults age 65 and older. (National Institute of Arthritis and Musculoskeletal and Skin Diseases, NIH, Bethesda Md.). RA is characterized by inflammation of the lining of the joints and/or other internal organs, and the presence of elevated numbers of lymphocytes and high levels of proinflammatory cytokines.

Anti-inflammatory agents used to treat RA traditionally include aspirin and non-steroidal anti-inflammatory drugs (NSAIDS), such as ibuprofen (Motrin™, Advil™), fenoprofen, indomethacin, naproxen (Naprosyn™, Alleve™), and others. These are widely used medications that are effective in relieving pain and inflammation associated with RA. However, the side effects associated with frequent use of many of these medications include life-threatening gastrointestinal bleeding and kidney damage. Similar drugs, called Cox-2 inhibitors, while effective, have recently encountered safety concerns regarding a risk of cardiovascular problems. Currently, there are three available—valdecoxib (Bextra™), rofecoxib (Vioxx™) and celecoxib (Celebrex™). The benefits from these medications may take weeks or months to be apparent. Because they are associated with possible adverse side effects, monitoring of patients, including blood tests, while on these medications is advised.

Other drugs that suppress the immune system, like azathioprine (Imuran™) and cyclophosphamide (Cytoxan™), are sometimes used in people who have failed other therapies. These medications are also associated with potentially significant side effects, and are usually reserved for severe cases of RA.

Corticosteroids have also been used to reduce inflammation in RA for more than 40 years. However, because of potential long-term side effects, corticosteroid use is usually limited to short courses and low doses where possible. Side effects may include bruising, psychosis, thinning of the bones (osteoporosis), cataracts, weight gain, and susceptibility to infections, diabetes, and high blood pressure.

Given the irreversibility of arthritis and the destruction of joints, tendons, ligaments and other tissues, it is desirable to provide a preventative modality to this debilitative disease. Accordingly, it is highly desirable to have a safe and efficacious vaccine that prevents or delays the onset and severity of arthritis symptoms in susceptible individuals. Ideally, such vaccinations will be provided to subjects that have been clinically diagnosed as susceptible or those subjects that are showing early signs of arthritis but do not have advanced clinical symptoms.

### SUMMARY

In various embodiments, the invention provides vaccine compositions for preventing the onset or induction of arthritis in a subject susceptible to developing arthritis. In particular, vaccines are provided comprising an arthritic protective peptide comprising the amino acid sequence of SEQ ID NO: 1. In one aspect of the invention, the anti-arthritic peptide is admixed with an adjuvant in a pharmaceutically acceptable carrier.

In another aspect, the present invention provides methods for preventing arthritis in a human or other mammal subject comprising administering to the subject an anti-arthritic peptide. In various embodiments, the subject is susceptible to the onset of arthritis. The invention further comprises methods of testing whether a subject is susceptible to developing arthritis. The susceptible subject is vaccinated with an effective amount of the anti-arthritic peptide.

In another aspect of the invention, kits are provided for carrying out the methods disclosed herein. Such kits comprise in one or more doses of an anti-arthritic peptide and instructions for administering the peptide.

It has been found that the compositions and methods of this invention are effective for preventing or delaying the onset of arthritis. These methods and compositions afford advantages versus compositions and methods among those known in the art, including one or more of enhanced vaccination efficacy, increased duration of action, and reduction of side effects.

### DETAILED DESCRIPTION

The following definitions and non-limiting guidelines must be considered in reviewing the description of this invention set forth herein.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific examples are provided for illustrative purposes of how to make, use and practice the compositions and methods of this invention and, unless explicitly stated otherwise, are not intended to be a representation that given embodiments of this invention have, or have not, been made or tested.

The headings (such as “Introduction” and “Summary,”) and sub-headings (such as “Vaccine Compositions”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

Unless otherwise indicated, the practice of the invention will employ conventional techniques of molecular biology, microbiology, recombinant DNA, and immunology, within the skill of the art. Such techniques are explained in the standard literature, such as: “Molecular Cloning: A Laboratory Manual”, Second Edition (Sambrook, Fritsch & Maniatis, 1989), “Oligonucleotide Synthesis” (M. J. Gait, ed., 1984), “Animal Cell Culture” (R. I. Freshney, ed., 1987); the series “Methods in Enzymology” (Academic Press, Inc.); “Handbook of Experimental Immunology” (D. M. Weir & C. C. Blackwell, Eds.), “Gene Transfer Vectors for Mammalian Cells” (J. M. Miller & M. P. Calos, eds., 1987), “Current Protocols in Molecular Biology” (F. M. Ausubel et al., eds., 1987); and “Current Protocols in Immunology” (J. E. Coligan et al., eds., 1991).

The present invention provides compositions and methods for the prevention of arthritis in human and other mammal subjects. Specific compounds and compositions to be used in the invention must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

Anti-Arthritic Peptide:

The present invention provides anti-arthritic peptides derived from sequences that have homology between porcine myelin basic protein (MBP) and measles nucleocapsid proteins for use in vaccine compositions and methods to prevent the onset of arthritis. The anti-arthritic peptides are capable of preventing and reducing symptoms of adjuvant induced arthritis, a disease most commonly associated with RA in humans (Henderson, B., Edwards, J. C. W., Pettipher, E. R., eds. Mechanisms and Models in RA. 1995. London: Academic Press) when administered as a vaccine composition.

In certain embodiments, the anti-arthritic peptides (herein “Anti-Arthritic Peptides”) of the invention are homologous to regions of MBP and measles nucleocapsid proteins including the stretch of amino acids spanning 110-162 of MBP. Peptides according to certain embodiments include peptide sequences from two to twelve amino acids, more preferably from five to fifteen amino acids, most preferably ten to twenty amino acids of SEQ ID NO: 1.: Met-Ser-Lys-Thr-Glu-Trp-Asn-Ala-Ser-Gln-Ser-Arg-Phe-Gly-Trp-Phe-Glu-Asn-Lys-Glu. The present invention further provides isolated, synthesized and recombinant peptides that are at least 77% homologous to a peptide set forth in SEQ ID NO: 1, more preferably 80% homologous, still more preferably 85% homologous, most preferably 90% homologous.

As used herein, the term “homologous” or its grammatical equivalents (including homologs, derivatives and variants) means that the peptide's homologous region must be a continuous sequence of at least ten amino acids achieving a “similarity score” (SS) of at least 77% (preferably higher). The “similarity score” (SS) is determined as follows. The regions to be compared are aligned to achieve a maximum score. The SS score is calculated by awarding each identical pair of amino acids in the aligned regions one point and each conserved pair of amino acids in that region half a point. The total points are added and divided by the total number of amino acid residues compared. By way of example, a 77% SS would equal a total score of 7.7 for a ten amino acid aligned pair of sequences, 15.4 for a twenty amino acid aligned pair of sequences, etc. At most one insertion or one deletion can be made in one of the sequences to maximize alignment scores. As used in calculating homology, conserved amino acids substitutions are defined in bracketed groups as follows: (Phe, Tyr, Trp); (Phe, Leu, Ile, Val); (Arg, Lys, His); (Glu, Asp, Ser); (Glu, Gln, Asp, Asn); (Ser, Thr); (Thr, Ala); and (Ala, Gly).

Anti-arthritic peptides useful herein may be made by a variety of methods, including methods for making peptides known in the art. Synthetic methods useful herein include such commonly used methods as t-BOC or FMOC protection of alpha-amino groups. Both methods involve stepwise syntheses whereby a single amino acid is added at each step starting from the C terminus of the peptide (see, Coligan, et. al., Current Protocols in Immunology, Wiley Interscience, 1991, Unit 9). Peptides of the invention can also be synthesized by various well known solid phase peptide synthesis methods, such as those described by Merrifield (J. Am. Chem. Soc., 85:2149, 1962), and Stewart and Young (Solid Phase Peptides Synthesis, Freeman, San Francisco, 1969, pp 27-62), using a copoly (styrene-divinylbenzene) containing 0.1-1.0 mmol amines/g polymer. On completion of chemical synthesis, the peptides can be deprotected and cleaved from the polymer by treatment with liquid HF-10% anisole for about ¼-1 hours at 0° C. Automated solid-phase peptide synthesis can be performed using devices such as a PE-Applied Biosystems 430A peptide synthesizer (commercially available from Applied Biosystems, Foster City, Calif.).

After evaporation of the reagents, the peptides are extracted from the polymer with 1% acetic acid solution which is then lyophilized to yield the crude material. This can normally be purified by such techniques as gel filtration on a “SEPHADEXE® G-15” or “SEPHAROSE®” affinity column using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and sequenced by the solid phase Edman degradation.

Anti-arthritic peptides can alternatively be produced recombinantly. Peptides of the present invention may be readily expressed in a variety of host cells or organisms. As used herein, a “host cell” refers to any cell that can be transfected or transformed with a suitable vector and will express the peptides encoded by the vector. In certain embodiments, it is desirable to produce a host cell which includes a nucleic acid encoding all or part of an anti-arthritic peptide within a recombinant expression vector or an anti-arthritic nucleic acid molecule containing sequences which allow it to homologously recombine into a specific site of the host cell's genome. A host cell can be any prokaryotic or eukaryotic cell. For example, an anti-arthritic peptide can be expressed in bacterial cells such as E. coli, insect cells, yeast, or mammalian cells (such as Chinese hamster ovary cells (CHO)) or COS cells. Other suitable host cells are known to those skilled in the art. Vector DNA can be introduced into host cells via conventional transformation or transfection techniques, e.g., any art-recognized technique for introducing foreign nucleic acid (e.g., DNA) into a host cell, including calcium phosphate or calcium chloride co-precipitation, DEAE-dextran-mediated transfection, lipofection, or electroporation. The host cells of the invention can be used to produce (i.e., express) an anti-arthritic peptide, e.g., by culturing a host cell (into which a recombinant expression vector encoding an anti-arthritic peptide has been introduced) in a suitable medium such that an anti-arthritic peptide is produced and optionally isolating an anti-arthritic peptide from the medium or the host cell.

In certain embodiments, a DNA sequence encoding a desired anti-arthritic peptide is introduced into an expression vector appropriate for the host. The DNA sequence is derived from an existing clone or synthesized. A preferred means of synthesis is amplification of the gene from cDNA, genomic DNA, or a recombinant clone using a set of primers that flank the coding region or the desired portion of the peptide. Restriction sites are typically incorporated into the primer sequences and are chosen with regard to the cloning site of the vector. If necessary, translational initiation and termination codons can be engineered into the primer sequences. The desired sequence can be codon-optimized for expression in a particular host. For example, a secreted form of a desired protein that is expressed in a fungal host, such as yeast, can be altered in nucleotide sequence to use codons preferred in yeast. Codon-optimization may be accomplished by methods such as splice overlap extension, site-directed mutagenesis, automated synthesis, and the like.

At minimum, the vector must contain a promoter sequence. Other regulatory sequences however can also be included. Such sequences include a transcription termination signal sequence, secretion signal sequence, (for example, mat-alpha or invertase genese), origin of replication, selectable marker, and the like. The regulatory sequences are operationally associated with one another to allow transcription or translation.

The plasmids used herein for expression of a desired peptide include a promoter designed for expression of the anti-arthritic peptides in a bacterial host. Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), mouse mammary tumor virus long terminal repeat (MMTV LTR), rous sarcoma virus long terminal repeat (RSV LTR), SV40, metallothionein promoter (see, e.g., U.S. Pat. No. 4,870,009) and other inducible promoters. For expression of the peptides, a promoter is inserted in operative linkage with the coding region of the desired protein or polypeptide. The promoter controlling transcription of the desired peptide may be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to the E. coli lacI repressor responsive to IPTG induction, the temperature sensitive λcI857 repressor, and the like. The E. coli lacI repressor is preferred.

In certain embodiments, the vector also includes a transcription terminator sequence. A “transcription terminator region” has either a sequence that provides a signal that terminates transcription by the polymerase that recognizes the selected promoter and/or a signal sequence for polyadenylation.

In certain embodiments, the vector is capable of replication in bacterial cells. Thus, the vector preferably contains a bacterial origin of replication. Preferred bacterial origins of replication include the f1-ori and col E1 origins of replication, especially the origin derived from pUC plasmids.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). Suitable markers for eukaryotes usually require a complementary deficiency in the host (e.g., thymidine kinase (tk) in tk-hosts). However, drug markers are also available (e.g., G418 resistance and hygromycin resistance).

The sequence of nucleotides encoding the desired anti-arthritic peptide can also include a classical secretion signal, whereby the resulting peptide is a precursor protein processed and secreted. The resulting processed anti-arthritic peptide can be recovered from the periplasmic space or the fermentation medium. Secretion signals suitable for use are widely available and are well known in the art (von Heijne, J. Mol. Biol. 184:99-105, 1985). Prokaryotic and eukaryotic secretion signals that are functional in E. coli (or other host) can be employed. The presently preferred secretion signals include, but are not limited to pelB, matα, extensin and glycine-rich protein.

One skilled in the art appreciates that there are a wide variety of suitable vectors for expression in bacterial cells and which are readily obtainable. Vectors such as the pET series (Novagen, Madison, Wis.) and the CMV mammalian expression vectors with tags available for simplified purification (Sigma-Aldrich, St. Louis, Mo.) are suitable for expression of a wide variety of proteins including anti-arthritic peptides of the present invention. In certain embodiments, a suitable plasmid is ampicillin resistant, has a colEI origin of replication, lacI^{q} gene, a lac/trp hybrid promoter in front of the lac Shine-Dalgarno sequence, a hexa-his coding sequence that joins to the 3′ end of the inserted gene, and an rrnB terminator sequence. The choice of a bacterial host for the expression of the desired peptide is dictated in part by the vector. Commercially available vectors are paired with suitable hosts. The vector is introduced in bacterial cells by standard methodology. Typically, bacterial cells are treated to allow uptake of DNA (for cloning DNA protocols, see generally, Sambrook et al., Sambrook et al., Molecular Cloning: A Laboratory Manual, Cold Spring Harbor Press, Vol 1-3. (1989). Alternatively, the vector may be introduced by electroporation, phage infection, or another suitable method.

The host cells used to manufacture the peptide of the present invention can include eukaryotic cells such as yeast cells, insect cells and mammalian cells. The principles that guide vector construction for bacteria as discussed above, are applicable to vectors for these organisms. In general, vectors are well known and readily available. Briefly, the vector should have at least a promoter functional in the host in operative linkage with the desired anti-arthritic peptide. Usually, the vector will also have one or more selectable markers, an origin of replication, a polyadenylation signal and transcription terminator.

Host cells comprising an anti-arthritic peptide expression vector may be cultured using standard media well known to the skilled artisan. The media will usually contain all nutrients necessary for the growth and survival of the cells. Suitable media for culturing E. coli cells include, for example, Luria Broth (LB) and/or Terrific Broth (TB). Suitable media for culturing eukaryotic cells include Roswell Park Memorial Institute medium 1640 (RPMI 1640), Minimal Essential Medium (MEM) and/or Dulbecco's Modified Eagle Medium (DMEM), all of which may be supplemented with serum and/or growth factors as necessary for the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

The amount of an anti-arthritic peptide produced by a host cell can be evaluated using established methods known in the art. Such methods include, without limitation, Western blot analysis, SDS-polyacrylamide gel electrophoresis, non-denaturing gel electrophoresis, High Performance Liquid Chromatography (HPLC) separation, immunoprecipitation, and/or activity assays such as DNA binding gel shift assays.

If an anti-arthritic peptide has been designed to be secreted from the host cells, the majority of peptide may be found in the cell culture medium. If however, the anti-arthritic peptide is not secreted from the host cells, it will be present in the cytoplasm and/or the nucleus (for eukaryotic host cells) or in the cytosol (for gram-negative bacteria host cells). For an anti-arthritic peptide situated in the host cell cytoplasm and/or nucleus (for eukaryotic host cells) or in the cytosol (for bacterial host cells), the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If an anti-arthritic peptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The solubilized anti-arthritic peptide can then be analyzed using gel electrophoresis, immunoprecipitation, or the like. If it is desired to isolate the anti-arthritic peptide, isolation may be accomplished using standard methods such as those described herein and in Marston et al., 1990, Meth. Enz., 182:264-75.

If inclusion bodies are not formed to a significant degree upon expression of an anti-arthritic peptide, then the peptide will be found primarily in the supernatant after centrifugation of the cell homogenate. The anti-arthritic peptide may be further isolated from the supernatant using methods such as those described herein.

The purification of an anti-arthritic peptide from solution can be accomplished using a variety of techniques. If the peptide has been synthesized such that it contains a tag such as Hexahistidine (anti-arthritic peptide/hexaHis) or other small peptide such as FLAG (Eastman Kodak Co., New Haven, Conn.) or myc (Invitrogen) at either its carboxyl- or amino-terminus, it can be purified in a one-step process by passing the solution through an affinity column where the column matrix has a high affinity for the tag. For example, polyhistidine binds with great affinity and specificity to nickel. Thus, an affinity column of nickel (such as the Qiagen.RTM. nickel columns) can be used for purification of anti-arthritic peptide/polyHis. See, e.g., Current Protocols in Molecular Biology. Section: 10.1.8 (Ausubel et al., eds., Green Publishers Inc. and Wiley and Sons 1993).

Additionally, anti-arthritic peptides can be purified through the use of a monoclonal antibody that is capable of specifically recognizing and binding to an anti-arthritic peptide. Other suitable procedures for purification include, affinity chromatography, immunoaffinity chromatography, ion exchange chromatography, molecular sieve chromatography, HPLC, electrophoresis (including native gel electrophoresis) followed by gel elution, and preparative isoelectric focusing. In some cases, two or more purification techniques may be combined to achieve increased purity.

Vaccine Compositions:

The present invention further provides a pharmaceutical composition comprising a safe and effective amount of an anti-arthritic peptide of this invention and a pharmaceutically acceptable carrier. A “safe and effective” amount of anti-arthritic peptide is an amount that is sufficient to have the desired vaccination effect in the human or other mammal subject, without undue adverse side effects (such as toxicity, irritation, or allergic response), commensurate with a reasonable benefit/risk ratio when used in the manner of this invention. The specific safe and effective amount of the anti-arthritic peptide will, obviously, vary with such factors as the particular condition, the physical condition of the patient, the nature of concurrent therapies and vaccinations (if any), the specific route of administration and dosage form, the carrier employed, and the desired vaccination regimen. The present invention provides vaccine compositions in unit dose form, in various embodiments, suitable for administration to a human or other mammal subject in a single dose. Such unit dose forms, in various embodiments, comprise from about 5 micrograms to about 500 milligrams of peptide, more preferably 10 micrograms to about 200 milligrams, most preferably 20 micrograms to 100 milligrams of peptide.

In various embodiments, the vaccine compositions of the present invention comprise (1) vaccine compositions comprising of purified anti-arthritic peptides, synthetic anti-arthritic peptides or recombinant anti-arthritic peptides; (2) vaccine compositions comprising anti-arthritic peptide and one or more anti-arthritic peptide variants; (3) vaccine compositions comprised of anti-arthritic peptide and one or more other peptide fragments of anti-arthritic peptide; or (4) vaccine compositions of plasmid or viral recombinant expression vectors encoding anti-arthritic peptides and variants thereof. For instance, anti-arthritic peptides may be introduced into a subject, including humans, linked to a carrier or as a homopolymer or heteropolymer of active peptide units. Alternatively, a “cocktail” of peptides can be used. In some embodiments, a mixture of more than one anti-arthritic variant peptide has the advantage of increased recognition by the target immune cells.

The peptides can be formulated into compositions comprising a pharmaceutically-acceptable carrier. The compositions can be administered to humans and animals either intramuscularly, intravenously, orally, locally (powders, ointments or drops), as a nasal spray or as a suppository.

Suitable pharmaceutical carriers may be in the form of physiologically acceptable sterile aqueous or non-aqueous solutions, dispersions, suspensions or emulsions and sterile powders for reconstitution into sterile injectable solutions or dispersions. In various embodiments, carriers comprise solvents, wetting agents, emulsifying and suspending agents. Examples of suitable aqueous and non-aqueous carriers include water, saline, ethanol, polyols (propyleneglycol, polyethyleneglycol, glycerol, hyaluronic acid, vegetable oils (such as olive oil), and injectable organic esters such as ethyl oleate, and mixtures thereof. Proper fluidity can be maintained, for example, by the maintenance of the required particle size in the case of dispersions and by the use of surfactants. The compositions may contain pharmaceutically acceptable auxiliary substances as required to approximate physiological conditions, such as buffering agents, tonicity adjusting agents such as sugars and salts, and wetting agents. Specific excipients include example, sodium acetate, sodium lactate, sodium chloride, potassium chloride, calcium chloride, sorbitan monolaurate, and triethanolamine oleate. Suspending agents useful herein include ethoxylated isostearyl alcohols, polyoxyethylene sorbitol and sorbitan esters, microcrystalline cellulose, aluminum metahydroxide, bentonite, agar-agar and tragacanth, and mixtures thereof.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral and suppository administration, a pharmaceutically acceptable nontoxic composition is formed by incorporating any of the normally employed excipients, such as those carriers previously listed, and generally from about 10% to about 95% of an anti-arthritic peptide, and more preferably at a concentration of from about 25% to about 75%.

In certain embodiments, anti-arthritic peptide vaccine compositions may be sustained- or controlled-delivery formulations. Techniques for formulating a variety of other sustained- or controlled-delivery means, such as liposome carriers, bio-erodible microparticles or porous beads and depot injections, are also known to those skilled in the art. See, e.g., International App. No. PCT/US93/00829, which describes the controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919 and European Patent No. 058481), copolymers of L-glutamic acid and gamma ethyl-L-glutamate (Sidman et al., 1983, Biopolymers 22:547-56), poly(2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent No. 133988). Sustained-release compositions may also include liposomes, which can be prepared by any of several methods known in the art. See, e.g., Eppstein et al., 1985, Proc. Natl. Acad. Sci. USA 82:3688-92; and European Patent Nos. 036676, 088046, and 143949.

In certain embodiments, vaccine compositions optionally comprise a safe and effective amount a pharmaceutically acceptable adjuvant. Such adjuvants are compounds, compositions, or biologic materials that are operable to increase the immungenicity of the anti-arthritic peptides of the present invention. Such adjuvants useful herein include those known in the art, such as incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, and combinations thereof. The effectiveness of an adjuvant may be determined by measuring the number of regulatory T-lymphocytes (Tregs) and arthritogenic CD4+T-lymphocytes circulating in the sites likely to be affected by arthritis which can include without limitation, the joint synovial fluid in subjects susceptible to RA.

Vaccination Methods:

The present invention provides methods for preventing arthritis in a human or other mammal subject by administering a safe and effective amount of an anti-arthritic peptide. As referred to herein, “preventing” refers to delaying or totally preventing the onset of symptoms of arthritis in a subject, such as in various embodiments a subject that is otherwise susceptible to arthritis.

For the purposes of this disclosure, the term “arthritis” will, unless context otherwise requires, refer to arthritides and related conditions involving immunologically induced inflammation of joints and tissues systemically, including the heart, lungs, kidneys, bones, muscles, blood vessels and skin and include, for example, without limitation: rheumatoid arthritis (“RA”), osteoarthritis, psoriasis, ulcerative colitis, Crohn's disease, ankylosing spondylitis, fibromyalgia, gout, systemic lupus erythematosus, scleroderma, and Sjögren's syndrome. Arthritis is commonly described as an autoimmune disease characterized by disregulated immune responses to self-antigens and is acerbated by chronic inflammation. Taking RA as a representative non-limiting arthritis disease, and without limiting the mechanism, function or utility of present invention, there are two common theories for the observed pathogenesis. Firstly, it is known that RA is associated with class II major histocompatibility antigens and a large number of CD4+ T-cells having skewed T-cell receptor gene usage is typical in RA. Secondly, another theory advanced, proposes that macrophages and synovial fibroblasts are effector cells of joint destruction, and it is also postulated that the priming step involves the aberrant autoimmune CD4+ T-cells.

The anti-arthritic peptide may be administered by any suitable route, by injection, usually intramuscularly or subcutaneously, orally by means of an enteric capsule or tablet, rectally as a suppository, as a nasal spray, by gene therapy and by other suitable routes of administration. For a human subject, a suitable dose of the anti-arthritic peptide depends, in part, upon the chosen route of administration and a number of other factors including the activity of the specific compound employed, the age, body weight, general health, sex, diet, time of administration, route of administration, and rate of excretion, drug combination, the severity of the particular disease undergoing therapy, and the judgment of the prescribing physician.

In various embodiments, the “effective” amount of an anti-arthritic peptide is that which is capable of inducing an arthritis-specific immunosuppresive response that prevents or delays the onset of arthritis after a single complete dose has been administered, or after several divided doses have been administered. In various embodiments, the anti-arthritic peptide is administered in the range of about 0.001 mg/kg to about 0.1 mg/kg, or alternatively in the range of about 5 micrograms to about 500 milligrams per inoculation. In some embodiments, methods comprise administering a single dose. In other embodiments, methods comprise administering several doses, which may be administered over a period of hours, days, months or years. For example, in various embodiments, a method comprises administering an initial dose of an anti-arthritic peptide, followed by administering a booster dose of the peptide. The booster is, in various embodiments, administered in the range of about 0.001 mg/kg to about 0.1 mg/kg, or alternatively in the range of about 5 micrograms to about 500 milligrams per inoculation.

Without limiting the mechanism, function or utility of present invention, the anti-arthritic peptide is effective in preventing a human or other mammal subject's cellular immune system from reacting to autoantigens and initiating the course and progression of arthritis. In another aspect of the invention, the anti-arthritic peptide down regulates the activity of the humoral response to arthritogenic antigens and thereby preventing the production of arthritis specific autoantibodies. The term “arthritogenic” refers to the presence of agents or conditions that would promote the onset, development, and progression of arthritis and associated sequelae. In various aspects of the invention, the method also includes generally a method to increase arthritis specific regulatory T-lymphocyte (Treg) activity.

In various embodiments, an anti-arthritic peptide is administered to a subject susceptible to developing arthritis. In certain embodiments, a subject diagnosed as being at risk for the onset of arthritis, and juveniles diagnosed as being at risk for the onset of seropositive RA, will be treated with one or more therapeutically effective dosages of an anti-arthritic vaccine composition. As referred to herein, subjects susceptible to developing arthritis include individuals with family members having a history with an arthritic disease; those who are otherwise predisposed to have arthritis; and those who have the initial physiological indicators of the disease, for example, have circulating autoantibodies specific to arthritis but do not yet have clinical symptoms. As used herein, the term “autoantibodies” are referred to circulating or tissue antibodies produced by the body, which bind to antigens that are normal bodily constituents.

In this sense, in some embodiments, subjects who are predisposed to the disease have Major Histocompatibility Complex-Human Leukocyte Antigens (MHC-HLA) which have been determined to be arthritis disease specific for example RA disease associated HLA-DRB1 alleles (HLA-DRB1*0401, HLA-DRB1*0404, HLA-DRB1*0408, HLA-DRB1*0101/2, HLA-DRB1*0405, HLA-DRB1*1402 and HLA-DRB1*10). (See Rosloniec et al., J. Immunol., (1998), 160:2573-2578, Patil et al., J. Immunol., (2001), 167: 7157-7168, and Gonzalez-Gay et al., Semin. Arthritis Rheum. (2002), 31(6): 355-360.) In addition to the presence of HLA haplotypes conferring arthritis specific HLA-DRB1 alleles, subjects who are deemed to be predisposed may also express arthritis specific autoantibodies. A subject may not be predisposed to develop the disease when such subject does not have a family history of arthritis but nevertheless express DRB1 HLA alleles and has no concomitant expression of autoantibodies. Subjects are also susceptible to developing the disease when one or more of the following autoantibodies are present: anti-rheumatoid factor (RF), anti-cyclic citrullinated peptide (anti-CCP), anti-keratin (AKA), anti-perinuclear factor (APF), anti-collagen type II (anti-CII).

To perform the antibody screening, a subject's blood sample can be taken and tested for the presence of arthritis specific autoantibodies. Specifically, antibodies described in the screening test can be evaluated by standard immunological screening techniques such as enzyme-linked immunosorbant assays (ELISA), by direct and indirect immunofluorescence, flow cytometry and by radiolabelled immuno assays (RIA), either single or double antibody techniques, and other techniques in which either the antibody combining site or the antigen is labeled with some detectable tag. See generally Maggio, Enzyme Immunoassay, CRC Press, Cleveland, Ohio (1981) and Goldman, M., Fluorescent Antibody Methods, Academic Press, New York, N.Y. (1980).

To perform the HLA typing step of the arthritis screening method, a biological sample, which may be any fluid or tissue likely to contain lymphocytes, but will preferably be whole peripheral blood or synovial fluid (the “lymphocyte sample”), will be obtained using conventional techniques from the individual to be screened for arthritis. To determine whether the individual's HLA molecules bear the DRB1 HLA molecules, conventional HLA typing techniques well known to those skilled in the art may be employed. It will be appreciated in this regard that the nucleotide and amino acid sequences for most known HLA antigens have been fairly well characterized and can be readily identified (see, e.g., Marsh, et al., Tissue Antigens, 37:181-189 (1991), and Bodmer, et al., Tissue Antigens, 37:97-105 (1991)). For example, with reference to the polynucleotide sequences reported by Marsh and Bodmer, et al., those of ordinary skill in the art will be able to construct oligonucleotide probes which will hybridize to target polynucleotide sequences for detection of specific HLA antigens in a lymphocyte sample. Techniques for construction of suitable probes and performance of hybridization techniques are described in greater detail infra and may be employed by those of ordinary skill in the art without undue experimentation. Commonly, however, HLA typing is performed serologically, i.e., by using standardized antisera (of defined specificity) to a lymphocyte sample together with complement and observing whether the test cells are killed. HLA typing may also be performed by other conventional immunological techniques such as the “Mixed Lymphocyte Reaction” wherein test lymphocytes are mixed with B lymphocytes of defined HLA specificity. In the Mixed Lymphocyte Reaction, test cells of specificity different than the B cells of known HLA type are stimulated and proliferate. Performance of all of these HLA typing techniques is well within the ordinary level of skill in the art.

In one embodiment, methods of the present invention comprise administering an anti-arthritic peptide to a human subject. In other embodiments, an anti-arthritic peptide is administered to another mammal subject. In certain embodiments, a subject is a companion animal. A “companion animal” herein is an animal of any species kept by a human owner or caregiver as a pet, or any animal of a variety of species that have been widely domesticated as pets, including dogs (Canis familiaris) and cats (Felis domesticus), whether or not the animal is kept solely or partly for companionship. Thus, “companion animals” herein include working dogs, farm cats kept for rodent control, etc., as well as pet dogs and cats.

For example, it is known that dogs, especially several larger breeds are most prone to arthritis: Golden Retrievers, Labrador Retrievers, German Shepherds, Newfoundlands and St. Bernards. These breeds of dogs are also known for developing a higher incidence of hip dysplasia as compared to other breeds, which are not prone to arthritis. Cats also experience severe and debilitative arthritis disease. For example, erosive polyarthritis in cats has been described as resembling RA in man. (See Pedersen, NC et al., Am. J. Vet. Res. (1980) 41(4): 522-535.) Equine arthritis is as complex and diverse as arthritis in humans. Horses (Equus caballus) are naturally prone to suffer osteoarthritis later in life. In some veterinary circles, arthritis in the horse is commonly associated with lameness and often reduces the horse's productive work years. One documented etiology of lameness occurs when an afflicted horse experiences “navicular disease.” Chronic navicular disease can be caused by arthritis of the navicular bone and associated structures. (March, L., “Navicular disease is a common lameness problem in horses.” University of Illinois, College of Veterinary Medicine (Feb. 06, 1995)). The present invention extends to vaccination compositions and methods of preventing the onset of arthritis in companion animals and other work animals for example without limitation, horses.

For administration to non-human animals, the anti-arthritic peptide or vaccine composition can, in addition to injection by parenteral administration, nasally or rectally, also be added to the animal feed or drinking water. In various embodiments, an animal feed or drinking water product may be formulated with a predetermined dose of the anti-arthritic peptide so that the animal takes in an appropriate quantity of the anti-arthritic peptide along with its diet. In various embodiments, a premix containing the anti-arthritic peptide may be added to the feed or drinking water to consumption by the animal.

In a certain embodiment of the invention, there is provided an anti-arthritic peptide vaccination kit at least one anti-arthritis vaccine composition according to the invention, and instructions for administering the anti-arthritic peptide vaccine. Optionally, the kit also comprises a syringe, sprayer, inhaler, or other device for administering the composition. In various embodiments, the kit comprises two or more doses of the peptide. In various embodiments, the kit additionally comprises a test, such as discussed above, for determining whether a subject is susceptible to arthritis.

In certain embodiments, the anti-arthritic peptides are administered to the subject or cells of the subject via gene therapy. In certain embodiments, anti-arthritic peptides are administered by delivering vectors to the subject, comprising nucleic acid sequences encoding anti-arthritic peptides of the present invention. Such nucleic acids, when transcribed and translated in a mammalian cell, encode the peptides of the present invention. In some embodiments, a nucleic acid encoding a peptide set forth in SEQ ID NO:1, or homologous variant thereof, within a recombinant vector is utilized. Such methods include those known in the art. Anti-arthritic peptide viral vectors can be particularly useful for delivering nucleic acids encoding anti-arthrits peptides of the invention to cells capable of secretion or surface expression of the peptides. Examples of vectors include those derived from influenza, adenovirus, vaccinia, herpes symplex virus, fowlpox, vesicular stomatitis virus, canarypox, poliovirus, adeno-associated virus, and lentivirus and sindbus virus. In addition, non-viral vectors, such as plasmids, cosmids, YACs and liposomes or even naked DNA, are also useful for delivering nucleic acids encoding anti-arthritic peptides of the invention to cells.

Nucleic acid molecules, such as those encoding anti-arthritic peptides, can be inserted into vectors and used as gene therapy vectors for genetic vaccination or ‘prime-boost’ vaccination regimes. A ‘prime-boost’ vaccination is a type of vaccination where administration of a genetic vaccine (such as a recombinant vector vaccine) is followed by a second type of vaccine (such as a protein subunit vaccine). The goal of ‘prime-boost’ vaccination is to stimulate different kinds of immune responses and enhance the body's overall immune response. Gene therapy techniques have recently become quite advanced and are meeting enviable success (Meikle, 2002). Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

In certain embodiments, methods for introducing a nucleic acid expressing a target nucleic acid sequence, for example a nucleic acid sequence encoding an anti-arthritic peptide of the present invention into immune cells can be accomplished by use of a viral vector. Examples of viral vectors which can be used include retroviral vectors (Eglitis, M. A., et al. (1985) Science 230:1395; Danos, O. and Mulligan, R. (1988) Proc. Natl. Acad. Sci., USA 85:6460); Markowitz, D., et al. (1988) J. Virol. 6:1120), adenoviral vectors (Rosenfeld, M. A., et al. (1992) Cell 68:143) and adeno-associated viral vectors (Tratschin, J. D., et al. (1985) Mol. Cell. Biol. 5:3251). Infection of immune cells with a viral vector has the advantage that a large proportion of cells will receive nucleic acid, thereby obviating a need for selection of cells which have received nucleic acid, and molecules encoded within the viral vector, e.g. by a cDNA contained in the viral vector, are expressed efficiently in cells which have taken up viral vector nucleic acid.

The nucleic acid sequences of this invention may be derived from a variety of sources including: cDNA, synthetic DNA, mRNA, synthetic RNA, or combinations thereof. The sequences may be obtained in any of several ways. For example, the polymerase chain reaction (PCR) method can be used to produce the nucleic acids of the invention, using either RNA (e.g., synthetic RNA) or DNA (e.g., cDNA and/or synthetic DNA) as templates. Primers used for PCR can be synthesized using the amino acid sequence SEQ ID NO:1 provided herein and can further be designed to introduce appropriate new restriction sites, if desirable, to facilitate incorporation into a given vector for recombinant expression.

In some embodiments, a vector comprising a nucleic acid encoding the anti-arthritic peptide (including one or more variants) is used to transfect a compatible host infected with vaccinia virus. Vaccinia virus recombinants packaged with the anti-arthritis encoded DNA is then grown and used as a viral vaccine. (Construction and characterization of vaccinia virus recombinants, Mackett, M. in DNA Cloning 4: A Practical Approach Mammalian Systems, 2^{nd} Ed. Eds: Glover, D. M and Hames, B. D. (1995). Non-viable recombinant vaccinia virus particles are then grown in culture and used to infect a subject or isolated immune cells obtained from the subject, for example: peripheral blood mononuclear cells (PMBCs). PMBC isolation and purification protocols are commonly known. (See Current Protocols in Immunology” (J. E. Coligan et al., eds., 1991). PMBCs include antigen presenting cells including dendritic cells monocytes and macrophages. When a vaccinia virus expressing anti-arthritic peptides on the surface act as “superantigens” capable of interacting with antigen-presenting cells (APC). The anti-arthritic peptide is then processed by the APCs and can then be presented to regulatory T cells which in turn become activated and secrete IL-10 and TGF-β thus suppressing the activity of self-reactive CD4+ T-cells capable of inducing or exacerbating the arthritic disease.

In certain embodiments, host cells presenting anti-arthritic peptide antigen on the surface of the host cell are used to stimulate the expansion of anti-arthritic peptide specific regulatory T lymphocytes ex vivo followed by introduction of the stimulated Tregs into a patient. For example purified PBMCs can be isolated from the subject and purified to remove contaminating red blood cells and other non-cellular constituents. PMBC are then incubated with cells that are previously infected with a compatible virus, for example Herpes Simplex Virus (HSV). The target cells (PMBCs) can be infected with the virus encoding a anti-arthritic peptide along with an internal ribosome entry site (IRES) and appropriate promoters for example the immediate early promoter of cytomegalovirus (CMV) or the SV40 early promoter. The HSV infected cells are then irradiated using gamma irradiation. The target PMBCs are then added in culture over the irradiated cells. The cells are then cultured for 24 hours and non-adherent PBMCs are then collected and injected into a subject as described in Brown et al. “Retroviral Vectors” in DNA Cloning 4: A Practical Approach Mammalian Systems, 2^{nd} Ed. Eds: Glover, D. M and Hames, B. D. (1995), pgs. 113-142. Foreign genes, for example, anti-arthritic peptides delivered via retroviruses can be in theory expressed in target cells for months.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. A vaccine comprising an arthritic protective peptide comprising the amino acid sequence of SEQ ID NO: 1.

2. A vaccine according to claim 1, wherein said protective peptide is isolated from a natural source, a synthetic peptide, or a recombinant peptide.

3. A vaccine according to claim 2, further comprising a second peptide sharing at least 77% homology to said anti-arthritic protective peptide.

4. A vaccine according to claim 1, comprising said peptide and an adjuvant in a pharmaceutically-acceptable carrier.

5. A method of preventing the onset of arthritis in a mammal, said method comprising a screening step to identify a mammal in need of an anti-arthritic vaccine according to claim 1, wherein said mammal in need having tested positive for one or more arthritis risk factors.

6. A method according to claim 5, wherein said arthritis risk factors is selected from the group consisting of anti-RF antibodies, anti-CCP antibodies, anti-keratin antibodies, anti-perinuclear factor antibodies, MHC-HLA antigens, consisting of HLA-DRB1*0401, HLA-DRB1*0404, HLA-DRB1*0408, HLA-DRB1*0101/2, HLA-DRB1*0405, HLA-DRB1*1402 and HLA-DRB1*10 and equivalents thereof.

7. A vaccine for preventing the onset of arthritis comprising an arthritic protective peptide and an adjuvant in a pharmaceutically acceptable carrier wherein said anti-arthritic protective peptide comprises a peptide set forth in SEQ ID NO: 1.

8. A method of treating arthritis in a mammal, said method comprising administering to a mammal an effective amount of an protective peptide comprising the peptide sequence of SEQ ID NO: 1.

9. A method according to claim 8, wherein said administering comprises administering a plurality of doses of said protective peptide over a period of time so as to inhibit the production of arthritogenic immune cells to a level below that existing prior to said administration.

10. A method according to claim 8, wherein said administration is selected from the group consisting of: intramuscular, subcutaneous, intravenous, oral, nasal, rectal and gene therapy.

11. A method according to claim 10, wherein said administration is subcutaneous.

12. A method according to claim 10, wherein said administration is by gene therapy.

13. A method according to claim 8, comprising administering a composition comprising said protective peptide and an adjuvant.

14. A method according to claim 8, wherein the mammal is human.

15. A method according to claim 8, wherein the mammal is a companion animal.

16. A method according to claim 8, wherein said protective peptide is administered at a dosage of at least 20 micrograms.

17. A method for suppressing the immune response of a human or other animal subject to arthritogenic epitopes comprising administering to said subject an effective anti-arthritic dose of a protective peptide comprising the amino acid sequence of SEQ ID NO: 1.

18. A method according to claim 17, wherein said administration is subcutaneous.

19. A method according to claim 17, wherein said protective peptide is administered at a level of from about 20 micrograms to about 500 milligrams.

20. A method according to claim 19, wherein said protective peptide is administered in unit dosage form, wherein the protective peptide comprises the amino acid sequence set forth in SEQ ID NO:1.

21. A method according to claim 17, further comprising administering at least one booster dosage of said protective peptide following said administration of said protective peptide.

22. A method according to claim 21, wherein the level of immune suppression to arthritogenic epitopes in said subject is determined through measurement of IL-10 prior to administering said booster dosage.

23. A method to prevent the secretion by immune cells of arthritis specific antibodies in a human or other animal subject, said method comprising administering to a mammal an effective amount of a protective peptide comprising the amino acid sequence of SEQ ID NO: 1, so as to stimulate the production of arthritogenic specific regulatory T-cells in said subject to a level above that existing prior to said administration.

24. A method according to claim 23, wherein the regulatory T-cells are CD4+CD25+ T-cells.

25. A method according to claim 23, wherein said protective peptide is administered at a dosage of from about 0.001 mgs/kg to about 0.1 mgs/kg.

26. A method according to claim 23, additionally comprising administering a booster dosage of said protective peptide following said administration if serum levels of said anti-protective peptide in said subject fall below about 0.001 mgs/kg.

27. A method to increase regulatory T-cells specific for arthritogenic epitopes to prevent the onset of arthritis in a human or other animal subject, said method comprising administering to said subject an effective amount of cells having DNA or RNA nucleotides coding for a protective peptide comprising the amino acid sequence of SEQ ID NO: 1, so as to stimulate the production of regulatory T-cells specific for arthritogenic epitopes in said subject to a level above that existing prior to said administration.

28. A method according to claim 27, additionally comprising administering to said human or other animal subject cells having been treated in vitro to insert therein a vector comprising a DNA segment encoding a protective peptide comprising the amino acid sequence of SEQ ID NO: 1, said cells expressing in vivo in said subject an effective amount of a protective peptide to prevent the onset of arthritis.


SEQUENCE LISTING (1 sequence)

SEQ ID NO: 1 - protein, 20 aa, Artificial
  Anti-Arthritic Protective Peptide 1 to 20 AA
MSKTEWNASQLPRLGGKEDR